(19) United States
(12) Patent Application Publication
(10) Pub. No.: US 2010/0179472 A1
(43) Pub. Date: Jul. 15, 2010

(54) Dynamic Pressure Gauged Breast Pump

(76) Inventor: Richard Scott Weston, Encinitas, CA (US)

Correspondence Address: Delaney IP, P.O. Box 9542, Rancho Santa Fe, CA, 92067, US

(21) Appl. No.: 12/354,762

(22) Filed: Jan. 15, 2009

(51) Int. Cl. A61M 1/06

(52) U.S. Cl. 604/67

(57) ABSTRACT

A breast pump system is disclosed for cyclically applying and simultaneously monitoring the negative pressure imposed while expressing milk from a breast. Structurally, the system includes a breast shield formed with a recess for receiving a breast. Further, a collection reservoir is connected to the recess of the breast shield for receiving milk therefrom. A pump is connected in fluid communication to the recess of the breast shield for applying the negative pressure to the breast. Also, a one-way valve is positioned between the recess and the collection reservoir to permit flow of milk into the collection reservoir while preserving ambient pressure in the collection reservoir. Importantly, a pressure gauge is connected between the pump and the breast shield for monitoring the operational pressure on the breast in real time. Further, a regulator manages the negative pressure applied by the pump in response to the monitored operational pressure.

### FIELD OF THE INVENTION

The present invention pertains generally to breast pumps. More particularly, the present invention pertains to systems and methods for monitoring pressures imposed by negative pressure breast pumps in real time. The present invention is particularly, but not exclusively, useful as a breast pump system that displays the operational pressure imposed on the breast while milk is expressed from the breast.

### BACKGROUND OF THE INVENTION

For various reasons, a lactating woman may desire to express and collect her milk. Accordingly, breast pumps have long been provided. Typically, breast pumps are used to allow a nursing mother to express beast milk to feed to her child at a later time when the mother may be absent. In other cases, the child is unable to nurse from the breast and may be fed breast milk by bottle. Also, a mother may have difficulties related to excessive breast milk production and require milk to be expressed despite normal nursing by her child. On the other hand, a mother may suffer from deficient milk production and require mechanical stimulation to express sufficient milk for her child.

Structurally, breast pumps typically include a funnel-type cup for receiving a breast. Further, the cup is operationally connected to a receiving bottle and to a source of suction. Both hand-operated and automatic designs exist, but for the automatic systems, an electric pump is provided to apply negative pressure to the breast.

Typically, electric breast pumps designed for extracting milk are concerned only with limiting their negative pressure to safe levels. For instance, many systems provided the ability to measure the pressure applied by breast pumps by positioning a cork in the receiving line and measuring the static pressure applied against the cork. However, as an inflexible static object, the cork may not represent the pressure that will be applied to a breast by the pump. Further, the output of the pump may vary during use. Therefore, a pressure reading obtained through measurement on a cork may not represent the actual pressure during a milk expressing procedure.

In light of the above, it is an object of the present invention to provide a breast pump having an integral dynamic pressure gauge. More specifically, it is an object of the invention to provide a system that incorporates a gauge between the pump and the recess of the breast shield to monitor the actual operational pressure on the breast during a milk expressing procedure. Still another object of the present invention is to provide a breast pump which displays the measured operational pressure during the milk expressing procedure. Another object of the present invention is to provide a breast pump system that allows real time monitoring of dynamic pressure on the breast in order to allow adjustment of the pump to a desired operational pressure. Yet another object of the present invention is to provide a dynamic pressure gauged breast pump system and method that is easy to implement, is simple to use, and is comparatively cost effective.

### SUMMARY OF THE INVENTION

In accordance with the present invention, a dynamic pressure gauged breast pump system is provided for expressing milk from a breast. Importantly, the system monitors the actual pressure on the breast during the expressing procedure, and displays that operational pressure in real time. Structurally, the system includes a flexible breast shield formed with a recess for receiving a breast. Further, the system includes a collection reservoir connected to the recess for receiving and storing milk from the breast. Importantly, the collection reservoir is removably connected to the breast shield. Specifically, the system may include a housing that forms the breast shield and recess. Further, the housing forms an outlet to the recess, and provides a valve in the outlet. Also, the housing forms an adaptor portion that is designed for selective connection to the reservoir.

In the present system, a pump is connected in fluid communication to the recess of the breast shield. As a result of this connection, the pump is able to cyclically apply a negative pressure to the recess. Importantly, a pressure gauge positioned in the fluid line between the pump and the recess of the breast shield. Due to this structure, the gauge can monitor a dynamic operational pressure on the breast while expressing milk from the breast. Further, the system includes a display mechanism that is connected to the pressure gauge. As the pressure gauge monitors the operational pressure on the breast, the display mechanism shows the monitored pressure in real time. Also, the system includes a control for operating the pump components. Specifically, the control may include an on/off switch and a mechanism for adjusting the negative pressure applied by the pump. In certain embodiments, a housing may contain the pump, pressure gauge, display mechanism, and control components.

In operation, a breast is inserted into the recess of the breast shield and the pump is operated to cyclically apply a negative pressure. Initially, the negative pressure pulls the breast tissue into closer contact with the breast shield. Further, the negative pressure may pull and slightly deform the breast. During the milk expressing procedure, the pump may apply a selected negative pressure according to a programmed pattern. While the negative pressure is applied, the pressure gauge simultaneously monitors the operational pressure on the breast. Further, the display mechanism shows the operational pressure in real time. As a result, the user may correlate the operational pressure value to the actual feeling of pressure on her breast. Also, the user may adjust the negative pressure applied. At the end of the milk expressing procedure, the valve can be opened to break the sealed fluid communication between the breast and the pump. Thereafter, the breast can be removed from the breast shield and the reservoir can be disconnected.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a cross-sectional illustration of the milk collection components and a schematic illustration of the automatic pump and gauge components of a dynamic pressure gauged breast pump system in accordance with an embodiment of the present invention; and

FIG. 2 is a cross-sectional illustration of the milk collection components and manual pump and gauge components of a dynamic pressure gauged breast pump system in accordance with an embodiment of the present invention.

### DESCRIPTION OF THE PREFERRED EMBODIMENTS

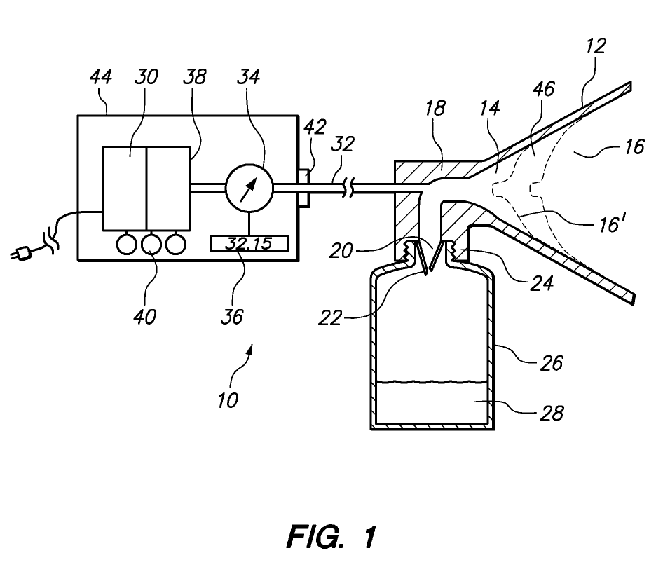

Referring initially to FIG. 1, a dynamic pressure gauged breast pump system is shown and generally designated 10. In FIG. 1, the system 10 includes a breast shield 12 that defines a recess 14 for receiving a breast 16. As shown, the breast shield 12 is formed by a housing 18. Structurally, the housing 18 forms an outlet 20 in fluid communication with the recess 14. Also, a one-way valve 22 is positioned in the outlet 20. As shown, the housing 18 further includes an adaptor portion 24 that selectively engages the outlet 20 to a collection reservoir 26 for receiving milk 28. Though exemplary, in FIG. 1 such engagement is attained via threaded connection.

Still referring to FIG. 1, the system 10 also includes an automatic pump 30 for cyclically applying a negative pressure to the recess 14. As shown, the pump 30 is powered by battery or through connection to an electrical supply via a cord and plug to create cyclically varying negative pressure. Further, the pump 30 is connected to the housing 18 via tubing 32 to establish fluid communication between the pump 30 and the recess 14. Importantly, a pressure gauge 34 is provided between, and in fluid communication with, the pump 30 and the recess 14. Further, a display mechanism 36, such as an analog or digital face, is connected to the pressure gauge 34. As a result, the cyclically-varying pressure between the pump 30 and the recess 14 may be measured and displayed in real time.

As shown in FIG. 1, a regulator 38 is connected to the pump 30 for managing the negative pressure applied by the pump in response to the cyclically-varying pressure measurement by the gauge 34. Further, control buttons 40 are also provided and are connected to the pump 30 and regulator 38. With these buttons 40, the pump 30 may be powered on or off as well as adjusted to provide a selected negative pressure. Also, a bleed valve 42 is provided along the tubing 32 to selectively release pressure between the pump 30 and the recess 14. In FIG. 1, it can be seen that a housing 44 holds the pump 30, pressure gauge 34, display mechanism 36, control buttons 40, and bleed valve 42.

In FIG. 1, a breast 16 is shown as being received within the breast shield 12 for a milk expressing procedure. Initially, there is a gap volume 46 between the breast 16 and the breast shield 12. As the pump 30 is activated, it applies an initial negative pressure to the recess 14. In response, the breast 16 is pulled more snugly into contact with the breast shield 12 and, in fact, deforms under the negative pressure as shown at 16′. As a result, a portion of the gap volume 46 is eliminated and sealed fluid communication is established between the pump 30, the breast 16 and the reservoir 26. Further, as the pump 30 applies negative pressure to the recess 14, the one-way valve 22 closes and the reservoir remains at ambient pressure. Therefore, the pump 30 need not remove air from the reservoir 26 to apply the selected negative pressure to the recess 14.

During the expressing procedure, the automatic pump 30 cyclically applies negative pressure to the recess 14. Specifically, the pump 30 applies negative pressure for one half second and then rests for one half second while the bleed valve 42 allows air to enter the tubing 32 to eliminate the negative pressure therein. During the half second that negative pressure is applied by the pump, milk 28 is released from the breast 16 and enters the recess 14. The milk 28 does not yet enter the reservoir 26 because the one-way valve 22 closes under the negative pressure regime. During the subsequent half second rest period, the negative pressure is released, the one-way valve 22 opens, and the milk 28 flows into the collection reservoir 26 under the force of gravity.

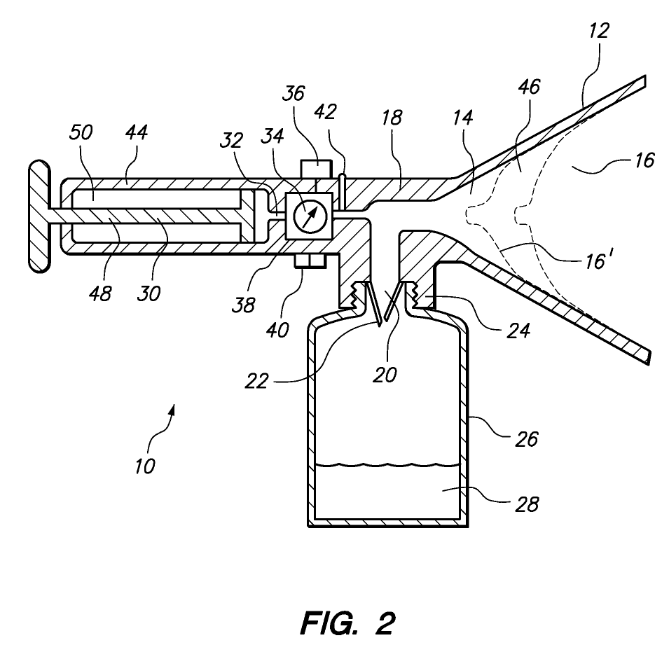

Referring now to FIG. 2, another embodiment of the dynamic pressure gauged breast pump system is shown and generally designated 10. Again, the system 10 includes a breast shield 12 defining a recess 14 that receives a breast 16. Structurally, a housing 18 forms the breast shield 12 and an outlet 20 in fluid communication with the recess 14. Also, a one-way valve 22 is positioned in the outlet 20. Further, the housing 18 includes an adaptor portion 24 that selectively engages the outlet 20 to a collection reservoir 26 for receiving milk 28.

In contrast to the system 10 shown in FIG. 1, the system 10 in FIG. 2 includes a manual pump 30 for cyclically applying a negative pressure to the recess 14. As shown, the pump 30 provides negative pressure through the axial movement of a piston 48 within an air-tight channel 50 formed in the housing 44. Further, the housing 44 holding the pump 30 is integral with or mounted to the housing 18 and a tubing 32 establishes fluid communication between the pump 30 and the recess 14. Importantly, a pressure gauge 34 is provided between, and in fluid communication with, the pump 30 and the recess 14. Further, a display mechanism 36, such as an analog or digital face, is connected to the pressure gauge 34. As a result, the cyclically-varying pressure between the pump 30 and the recess 14 may be measured and displayed in real time.

As shown in FIG. 2, a regulator 38 is connected to the pump 30 for managing the negative pressure applied by the pump in response to the cyclically-varying pressure measurement by the gauge 34. Further, control buttons 40 are also provided and are connected to the pump 30 and regulator 38. With these buttons 40, the pump 30 may be adjusted to provide a selected negative pressure. Also, a bleed valve 42 is provided along the tubing 32 to selectively release pressure between the pump 30 and the recess 14.

During the expressing procedure, the manual pump 30 applies negative pressure to the recess 14 in user-determined cycles. Specifically, the manual pump 30 applies negative pressure during the withdraw movement of the piston 48 relative to the channel 50. The duration of the negative pressure regime may be extended by holding the piston 48 at its withdrawn position. Thereafter, the negative pressure is released by inserting the piston 48 back into the channel 50. As a result, the user may determine the duration of the negative pressure and rest cycles.

In certain embodiments, negative pressure may be applied for one half second and then released for one half second while the negative pressure is dissipated. During the half second that negative pressure is applied by the pump, milk 28 is released from the breast 16 and enters the recess 14. The milk 28 does not yet enter the reservoir 26 because the one-way valve 22 closes under the negative pressure regime. During the subsequent half second rest period, the negative pressure is released, the one-way valve 22 opens, and the milk 28 flows into the collection reservoir 26 under the force of gravity.

For systems 10 with either the automatic pump of FIG. 1 or the manual pump of FIG. 2, the pressure gauge 34 is able to monitor the operational pressure on the breast 16 in real time, simultaneous with the expressing procedure despite the changing operational parameters in the system 10 during the milk expressing procedure. Further, the display mechanism 36 is able to display the monitored operational pressure. As a result, the user expressing milk 28 is able to read the actual negative pressure on her breast 16 and correlate it to the feeling of pressure on her breast 16. Further, the control buttons 40 provide the user with the ability to slightly adjust the pressure and to continue to identify the feeling on her breast 16 in view of the displayed pressure. At the end of the milk expressing procedure, the sealed fluid communication can be interrupted by opening the bleed valve 42. Thereafter, the breast shield 12 can be removed from the breast 16, and the reservoir 26 can be disconnected from the housing 18.

While the particular Dynamic Pressure Gauged Breast Pump as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A dynamic pressure gauged breast pump system comprising:
a breast shield formed with a recess for receiving a breast;
a collection reservoir connected to the recess of the breast shield for receiving milk therefrom;
a pump connected in fluid communication to the recess of the breast shield for cyclically applying a negative pressure to the recess;
a one-way valve interconnected between the collection reservoir and the recess to permit milk to flow into the collection reservoir from the recess while preserving ambient pressure in the reservoir;
a pressure gauge connected between the pump and the breast shield for monitoring a dynamic operational pressure on the breast while expressing milk from the breast; and
a regulator for managing the negative pressure applied by the pump in response to the monitored dynamic operational pressure.

2. A system as recited in claim 1 further comprising a means for displaying the operational pressure during the expressing procedure.

3. A system as recited in claim 1 further comprising a control for manually adjusting the negative pressure applied by the pump.

4. A system as recited in claim 1 further comprising:
a means for displaying the operational pressure during the expressing procedure;
a control for manually adjusting the negative pressure applied by the pump;
a bleed valve for releasing pressure between the pump and the breast; and
a housing containing the pump, gauge, regulator, display means, control and bleed valve.

5. A system as recited in claim 4 further comprising a housing forming the breast shield and the recess, wherein the housing is adapted for selective connection to the collection reservoir.

6. A system as recited in claim 1 wherein a gap volume exists between the breast and the breast shield, and wherein the negative pressure reduces the gap volume between the breast and the breast shield.

7. A system as recited in claim 1 wherein the pump applies negative pressure for one half second and rests for one half second during each cycle.

8. A system as recited in claim 1 wherein the pump is manually operated.

9. A breast pump system comprising:
a recess for receiving a breast to express milk therefrom;
a means for collecting the milk, said collecting means being in sealed fluid communication with the recess;
a pump connected in sealed fluid communication to the recess for cyclically applying a negative pressure to the breast; and
a one-way valve interconnected between the collecting means and the recess to permit milk to flow into the collecting means from the recess while preserving ambient pressure in the collecting means;
a dynamic pressure gauge connected between the pump and the recess for monitoring a dynamic operational pressure on the breast while expressing milk from the breast; and
a regulator for managing the negative pressure applied by the pump in response to the monitored dynamic operational pressure.

10. A system as recited in claim 9 further comprising a means for displaying the operational pressure during the expressing procedure.

11. A system as recited in claim 10 wherein the displaying means is digital, and displays the operational pressure in real time.

12. A system as recited in claim 9 further comprising a control for manually adjusting the negative pressure applied by the pump.

13. A system as recited in claim 9 further comprising:
a means for displaying the operational pressure during the expressing procedure;
a control for manually adjusting the negative pressure applied by the pump;
a bleed valve for releasing pressure between the pump and the breast; and
a housing containing the pump, gauge, regulator, display means, control and bleed valve.

14. A system as recited in claim 9 further comprising a housing forming the recess, wherein the housing is adapted for selective connection to the collecting means.

15. A system as recited in claim 14 wherein a gap volume exists between the breast and the housing, and wherein the negative pressure reduces the gap volume between the breast and the housing.

16. A system as recited in claim 9 wherein the pump applies negative pressure for one half second and rests for one half second during each cycle.

17. A system as recited in claim 9 wherein the pump is manually operated.

18. A method for expressing milk from a breast comprising the steps of:
providing a dynamic pressure gauged breast pump system including a breast shield formed with a recess, a collection reservoir connected to the recess of the breast shield, a pump connected in fluid communication to the recess of the breast shield, a one-way valve interconnected between the collection reservoir and the recess, a pressure gauge connected between the pump and the breast shield, and a regulator;
positioning a breast in the recess of the breast shield;
operating the pump to cyclically apply a negative pressure to the breast to express milk therefrom, wherein the breast contacts the breast shield in response to the negative pressure to establish sealed fluid communication between the breast and the pump, and wherein the one-way valve permits milk to flow into the collection reservoir from the recess while preserving ambient pressure in the reservoir;
monitoring a dynamic operational pressure on the breast with the gauge while expressing milk from the breast; and
managing the negative pressure applied by the pump in response to the monitored dynamic operational pressure.

19. A method as recited in claim 18 further comprising the step of displaying the dynamic operational pressure in real time during the expressing procedure.

20. A method as recited in claim 18 wherein a gap volume exists between the breast and the breast shield, wherein the negative pressure reduces the gap volume between the breast and the breast shield, and wherein the dynamic operational pressure monitored by the pressure gauge compensates for the reduction in the gap volume.
